(19) United States
(12) Patent Application Publication
(10) Pub. No.: US 2023/0032650 A1
(43) Pub. Date: Feb. 2, 2023

(54) WEARABLE DEVICE AND OPERATION METHOD THEREOF

(71) Applicant: Po-Kang Lin, Taipei City (TW)

(72) Inventor: Po-Kang Lin, Taipei City (TW)

(21) Appl. No.: 17/873,196

(22) Filed: Jul. 26, 2022

(30) Foreign Application Priority Data
Jul. 29, 2021 (TW) 110127872

(51) Int. Cl. A61L 9/20; G02C 11/04; H05B 47/105

(52) U.S. Cl. CPC A61L 9/20; G02C 11/04; H05B 47/105; A61L 2209/111

(57) ABSTRACT

The present invention provides a wearable device including: frames; at least one UV light source; a left temple; a right temple; a detection device, used to detect a user's breathing; a control module electrically connected to the detection device, the at least one UV light source, and the power supply device. When the detection device detects that the user inhales, it transmits an activation signal to the control module, which activates the at least one UV light source by the activation signal so that the at least one UV light source emits UV light to the user's face. After the at least one UV light source is activated, when the detection device detects that the user exhales, it transmits a deactivation signal to the control module, which deactivates the at least one UV light source by the deactivation signal. Also, an operation method of a wearable device is provided.

### CROSS-REFERENCE TO RELATED APPLICATION

This application claims the priority of Taiwanese patent application No. 110127872, filed on Jul. 29, 2021, which is incorporated herewith by reference.

### BACKGROUND OF THE INVENTION

### 1. Field of the Invention

The present invention is directed to a wearable device and an operation method thereof, and more particularly to a wearable device including an UV light source and an operation method thereof.

### 2. The Prior Arts

In response to the current pandemic era, people will wear eyeglasses, goggles, and masks to reduce droplet infection from person to person, but in addition to the basic goggles and masks to reduce droplet infection, there is no further methods to block droplets and bacteria, which leads to the users' anxiety about blocking ability of the goggles or masks.

On the other hand, in the prior art, an UV source is provided on a wearable device, and a user's face region is sterilized by the UV light source. However, at present, the UV light source is normally on or off, so that the UV light source cannot provide an efficient sterilization and disinfection effect.

### SUMMARY OF THE INVENTION

It can be seen from the above-mentioned prior art that the UV light source currently provided on goggles or glasses can only be switched on or off normally. Therefore, there is a need to provide a wearable device with a different structure that not only provides a sterilization and disinfection effect, but also enables the UV light source to perform an efficient sterilization and disinfection operation.

A wearable device comprising: frames comprising a left frame and a right frame, the left frame being connected to the right frame by a bridge; at least one UV light source arranged on the left frame and/or the right frame; a left temple connected to the left frame; a right temple connected to the right frame; and a power supply device arranged in the frames, the left temple or the right temple, the power supply device being electrically connected to the at least one UV light source, wherein the at least one UV light source emits UV light to an area around a face of a user, wherein the at least one UV light source is arranged at a lower left side of the left frame and/or a lower right side of the right frame, and an included angle is provided between the at least one UV light source and a left side edge of the left frame and/or a right side edge of the right frame, the included angle being any angle in the range of 10-60 degree, irradiating the UV light source to a position around the nose and the mouth of the user.

Furthermore, the wearable device of the present invention further comprises a control module, which is arranged in the frames, the left temple or the right temple, and is electrically connected to the power supply device and the at least one UV light source, controlling the at least one UV light source to irradiate at a preset frequency.

The preset frequency is any second in the range of 1/15 second to 1/30 second.

In addition, the wearable device of the present invention further comprises a switch module, which is arranged on the frames, the left temple, or the right temple, and is electrically connected to the power supply device and the at least one UV light source, controlling a switch of the at least one UV light source.

The present invention also provides a wearable device comprising: frames comprising a left frame and a right frame, the left frame being connected to the right frame by a bridge; at least one UV light source arranged at a lower left side of the left frame and/or a lower right side of the right frame; a left temple connected to the left frame; a right temple connected to the right frame; a detection device arranged on the left frame, the right frame, the left temple, or the right temple; a control module arranged in the frames, the left temple, or the right temple, and electrically connected to the detection device and the at least one UV light source, and a power supply device arranged in the frames, the left temple, or the right temple, and electrically connected to the at least one UV light source, the detection device, and the control module, wherein the detection device is used to detect breathing of a user wearing the wearable device, when the detection device detects that the user inhales, the user exhales, the user is at end-exhalation, or at end-inhalation, the detection device transmits an activation signal to the control module, the control module activates the at least one UV light source by the activation signal, so that the at least one UV light source emits UV light to an area around a face of the user, wherein after the at least one UV light source is activated, the detection device transmits an deactivation signal to the control module when the detection device detects that the user exhales, the user inhales, the user is at the end-inhalation or at the end-exhalation, the control module deactivates the at least one UV light source by the deactivation signal.

Preferably, there is a included angle between the at least one UV light source and the left side edge of the left frame and/or the right side edge of the right frame, the angle being any angle in the range of 10-60 degree, so that the UV light source irradiates to a position around the nose and the mouth of the user.

Furthermore, the wearable device of the present invention further comprises a switch module, the switch module is arranged on the frames, the left temple or the right temple, and the switch module is electrically connected to the power supply device, and the switch module controls a switch of the power supply device.

Preferably, the detection device comprises an adjustment mechanism by which the detection device is rotated.

Preferably, the at least one UV light source is at least one deep UV light source.

In another aspect, the present invention also provides an operation method of a wearable device comprising the steps of: detecting breathing of a user wearing the wearable device by a detection device on the wearable device; transmitting an activation signal to a control module on the wearable device by the detection device when the detection device detects that the user inhales, the user exhales, the user is at end-exhalation or end-inhalation; activating at least one UV light source on the wearable device by a activation signal of the control module, so that the at least one UV light source emits UV light to an area around a face of the user; after the at least one UV light source is activated, transmitting an deactivation signal to the control module by the detection device when the detection device detects that the user exhales, the user inhales, the user is at end-inhalation or end-exhalation; and deactivating the at least one UV light source by a deactivation signal of the control module.

Moreover, the operation method of the present invention further comprises the steps of: adjusting an irradiation angle of the at least one UV light source by the user, so that the at least one UV light source irradiates to a position around the mouth and the nose of the user.

Moreover, the operation method of the present invention further comprises the steps of: activating or deactivating a power device on the wearable device by a switch module on the wearable device.

Preferably, the detection device is rotated by an adjustment mechanism.

Preferably, the at least one UV light source is at least one deep UV light source.

As can be seen from the above, the present invention provides a wearable device and operation method thereof. The wearable device of the present invention emits UV light to an area around the user's face and sterilizes the area around the user's face with the UV light. Further, the wearable device of the present invention can emit UV light to the area around the face of the user in conjunction with the user's breathing, and efficiently sterilize and disinfect the area around the face of the user by the UV light. More in detail, when the user has worn the mask and covers his/her nose and mouth with the mask, the wearable device can emit UV light in response to the user's breath and sterilize and disinfect the mask and an area in front of the mask, so that the bacteria attached to the mask can be eliminated and the user can be reassured. Thus, the wearable device of the present invention not only provides a substantial droplet barrier to protect the user's eye area, but also further provides efficient sterilization and disinfection of the area around the user's face. Further, according to the present invention, the irradiation mode can be adjusted according to the user's health condition so that the UV light source can sterilize and disinfect the air to be inhaled or exhaled.

### BRIEF DESCRIPTION OF THE DRAWINGS

The various aspects of the present invention as well as specific features and advantages thereof, will become more fully apparent to those having ordinary skill in the art upon reading the following detailed description and upon reference to the accompanying drawings, wherein:

FIG. 1 is a schematic diagram of the structure of a wearable device according to an embodiment of the present invention.

FIG. 2a is a schematic diagram of a wearable front configuration of a wearable device according to an embodiment of the present invention.

FIG. 2b is a schematic diagram of a wearable front configuration of a wearable device according to an embodiment of the present invention.

FIG. 3a is a schematic diagram of a wearable front configuration of a wearable device according to another embodiment of the present invention.

FIG. 3b is a schematic diagram of a wearable side configuration of a wearable device according to another embodiment of the present invention.

FIG. 4 is a schematic diagram of the structure of the wearable device according to further embodiment of the present invention.

FIG. 5 is a flowchart of an operation method of a wearable device according to an embodiment of the present invention; and

FIG. 6 is a flowchart of an operation method of a wearable device according to another embodiment of the present invention.

### DETAILED DESCRIPTION OF THE PREFERRED EMBODIMENT

Reference will now be made in detail to the present embodiments of the invention, examples of which are illustrated in the accompanying drawings and described in the accompanying drawings.

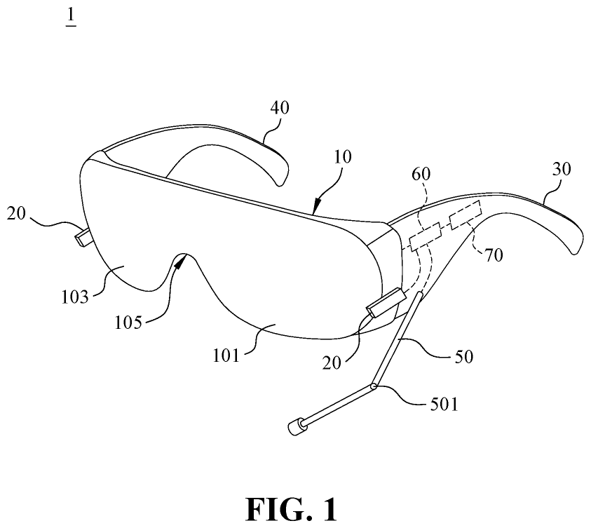

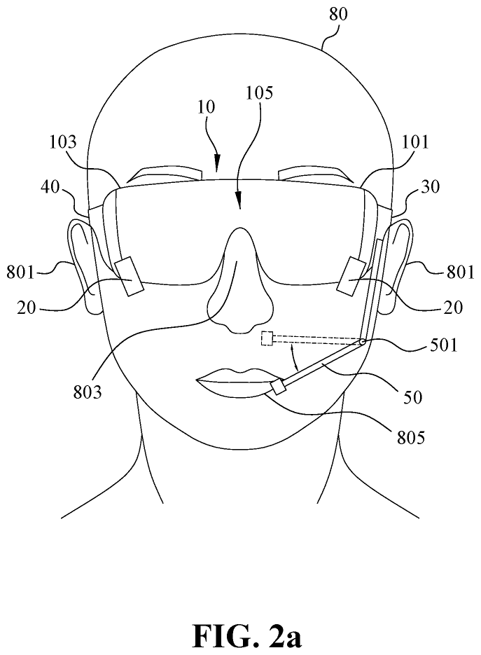

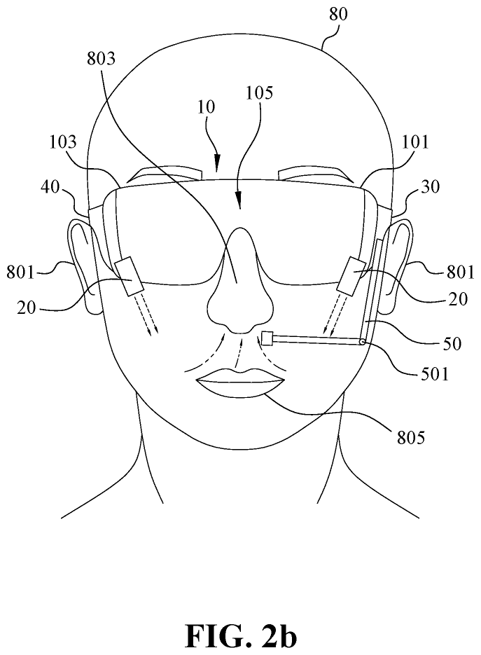

FIG. 1 is a schematic diagram illustrating a structure of a wearable device according to an embodiment of the present invention; FIG. 2a is a schematic diagram illustrating a wearable front structure of a wearable device according to an embodiment of the present invention; FIG. 2b is a schematic diagram of a wearable front structure of a wearable device according to an embodiment of the present invention. Referring to FIGS. 1, 2a, and 2b, in one embodiment of the present invention, a wearable device 1 comprises: frames 10, at least one UV light source 20, a left temple 30, a right temple 40, a detection device 50, a control module 60, and a power supply device 70.

Frames 10 comprise a left frame 101 and a right frame 103, the left frame 101 is connected to the right frame 103 via a bridge 105; at least one UV light source 20 is arranged at the lower left side of the left frame 101 and/or the lower right side of the right frame 103; a left temple 30 is connected to the left frame 101; the right temple 40 is connected to the right frame 103; a detection device 50 is arranged on the left frame 101, the right frame 103, the left temple 30, or the right temple 40. In FIG. 1, the detection device 50 is arranged on the left temple 30; the control module 60 is arranged in the frames 10, the left temple 30 or the right temple 40, and FIG. 1 shows that the control module 60 is arranged in the left temple 30, and the control module 60 is electrically connected to the detection device 50 and at least one UV light source 20; a power supply device 70 is provided in the frames 10, the left temple 30, or the right temple 40, and FIG. 1 shows that the power supply device 70 is provided in the left temple 30, and the power supply device 70 is electrically connected to at least one UV light source 20, the detection device 50 and the control module 60.

On the other hand, the UV light source 20 may be arranged on the left frame 101 and/or the right frame 103 in a snap-fit manner, a hanging manner, a clamping manner, an adhesive manner, or other arrangements. Furthermore, the UV light source 20 can be a deep ultraviolet light source (Ultraviolet-C, UVC), and the wavelength of the light emitted by the deep UV light source can be 100-300 nm, 280 nm, 275 nm, or 274 nm, and these wavelengths of UV light have an optimal disinfection and sterilization effect without damaging the skin. A user 80 can hang the left temple 30 and the right temple 40 of the wearable device on the ears 801 and the bridge 105 thereof is hung on the nose 803.

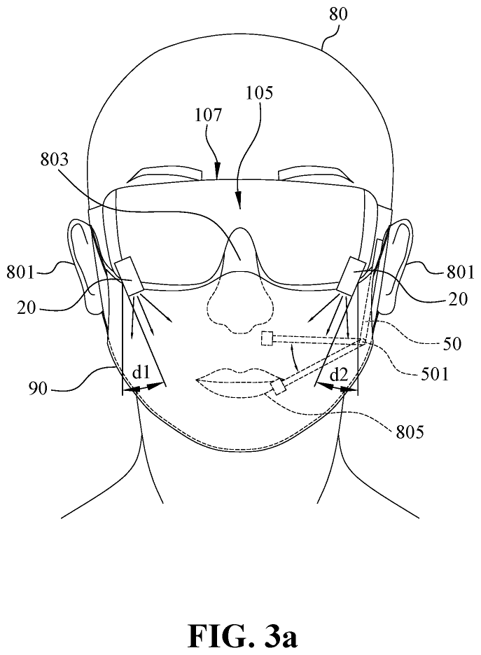

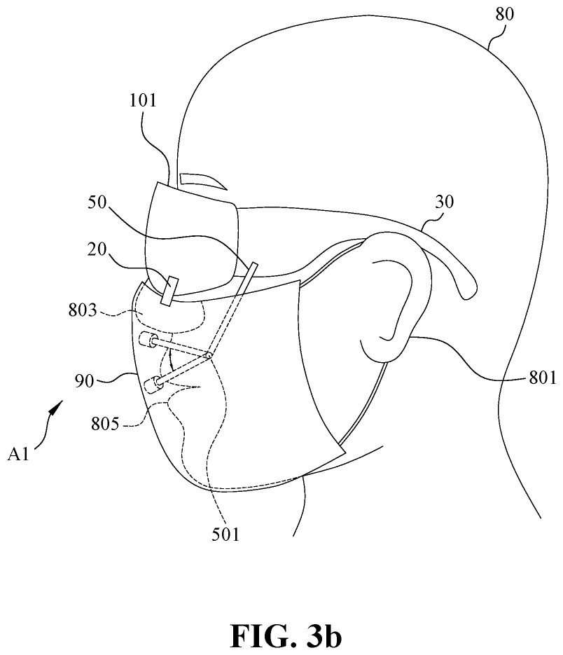

It is worth mentioning that the detection device 50 is used to detect breathing of the user 80 of the wearable device 1. When the detection device 50 detects that the user 80 inhales, the detection device 50 transmits an activation signal to the control module 60, and the control module 60 activates the UV light source 20 by the activation signal, so that the UV light source 20 emits UV light to an area A1 around the face of the user 80 (as shown in FIG. 3b), and after the at least one UV light source is activated, when the detection device 50 detects that the user 80 exhales, the detection device 50 transmits an deactivation signal, and the control module 60 deactivates the UV light source 20 by the deactivation signal.

In this way, the UV light source 20 of the present invention can efficiently sterilize and disinfect the area A1 around the face in response to breathing of the user 80 by the detection device 50, instead of only maintaining on or off state, and this embodiment also allows the wearable apparatus 1 to save power. The detection device 50 may be a microphone, particle detector or another type of detection device, such as voice detection device or air particle monitor.

On the other hand, although FIG. 2b shows that the UV light is emitted upon detection of inhalation by the user 80, in other embodiments, the UV light may be emitted by a different detection means. More in detail, when the detection device 50 detects that the user 80 is at the end-exhalation, the detection device 50 transmits an activation signal to the control module 60, and the control module 60 activates the UV light source 20 by the activation signal, so that the UV light source 20 emits UV light to an area A1 (shown in FIG. 3b) around the face of the user 80, and after the at least one UV light source is activated, when the detection device 50 detects that the user 80 is at end-inhalation, the detection device 50 transmits a deactivation signal to the control module 60, and the control module 60 deactivates the UV light source 20 by the deactivation signal. Such an irradiation mode allows the user 80 to sterilize and disinfect the air before the user 80 is ready to inhale.

Further, the above-mentioned ultraviolet irradiation method is directed to a healthy user so that the air inhaled by the healthy user can be sterilized by UV light every time. It is to be understood that the present invention can also adjust the irradiation mode so that the UV irradiation is directed to a user with pathogens; thereby the air exhaled by the user with pathogens can be sterilized and disinfected by UV light each time. For example, when the detecting device 50 detects that the user 80 exhales or is at end-inhalation, the detecting device 50 transmits an activation signal to the control module 60, and the control module 60 activates the UV light source 20 by the activation signal, so that the UV light source 20 emits UV light to an area A1 (shown in FIG. 3b) around the face of the user 80, and after the at least one UV light source is activated, when the detecting device 50 detects that the user 80 inhales or is at end-exhalation, the detecting device 50 transmits an deactivation signal to the control module 60, and the control module 60 deactivates the UV light source 20 by the deactivation signal. In this manner, gases exhaled by the user with pathogens can be sterilized and disinfected by irradiating to the UV light source 60 to reduce the probability of infecting others with the exhaled air.

From the above-described irradiation mode, it can be seen that the detection device 50 of the present invention can adjust the irradiation mode of the UV light source 20 by detecting breathing of the user. Since the exhalation and inhalation of the human body have a specific frequency, a specific audio frequency, and a sound level, the detection device 50 and the control module 60 can detect whether the exhalation and inhalation of the user 80 are at end-exhalation or at end-inhalation by using the above-mentioned characteristics.

Additionally, in other embodiments, the detection device 50 may include an adjustment mechanism 501 by which the detection device 50 may be rotated such that the detection device detects the nose 803 or the mouth 805 of the user 80. Thus, whether the user 80 is breathing through the nose 803 or the mouth 805, the detection device 50 may adjust and detect breathing.

Further, the UV light source 20 may be arranged only on the left frames 101 or the right frame 103, or may be arranged on both the left frame 101 and the right frame 103. In addition, although one power supply device 70 is shown in FIG. 1, the power supply device 70 may be selectively arranged in the frames 10, the left temple 30 or the right temple 40, or may be arranged in combination with the number of UV light sources 20.

FIG. 3a is a schematic diagram illustrating a wearable front structure of a wearable device according to another embodiment of the present invention; FIG. 3b is a schematic diagram illustrating a wearable side structure of a wearable device according to another embodiment of the present invention. Referring to FIGS. 3a and 3b, in another embodiment of the present invention, the user 80 can hang the left temple 30 and the right temple 40 of the wearable device on the ears 801, and the bridge 105 thereof is hung on the nose 803. The user wears a mask 90. In this embodiment, when the user 80 wears the mask 90, the detection device 50 is placed inside the mask 90 so that wearing the mask 90 does not interfere with the operation of the detection device 50, which instead supports the mask 90 and provides breathing space for the user 80.

Further, when the user 80 wears the mask 90 and covers the nose 803 and the mouth 805 by the mask 90, the detection means 50 controls the UV light source 20 to emit UV light in response to breathing of the user 80 and sterilizes and disinfect the mask 90, thereby eliminating the bacteria attached to the mask 90 and making the user 80 assured. Therefore, the wearable device 1 of the present invention can not only provide a basic droplet blocking capability, but also further provide a sterilization and disinfection effect to the area A1 around the face of the user 80, and further enable the UV light source 20 to be activated and deactivated by means of the detecting device 50, thereby enabling the UV light source 20 to perform power-saving and efficient light emission. It should be understood that the area A1 around the face of the user 80 can be sterilized and disinfected by the UV light emitted from the UV light source 20 even if the user 80 does not wear the mask 90.

On the other hand, in the embodiments shown in FIGS. 2a and 3a, the UV light source 20 is respectively arranged at the lower right side of the right frame 103 and the lower left side of the left frame 101, and there is an included angle d1 between the UV light source 20 and the right side edge of the right frame 103, and there is an included angle d2 between the other UV light source 20 and the left side edge of the left frame 101, wherein the included angle d1 and the included angle d2 can be any angle in the range of 10-60 degree, so that the UV light source 20 irradiates to positions around, in front of and above the mouth and nose in the area A1 around the face of the user 80; so as to enhance the sterilization and disinfection effect against the positions around the mouth and nose of the user 80.

In this embodiment, the UV light source 20 is arranged at the lower right side of the right frame 103 and the lower left side of the left frame 101, respectively. However, it should be understood that the UV light source 20 may be arranged only at the lower left side of the left frame 101 or at the lower right side of the right frame 103, or at both the lower right side of the right frame 103 and the lower left side of the left frame 101, and need not be angled with respect to the left side edge and/or the right side edge. The arrangement in this embodiment further prevents the path of UV light from passing through the eye.

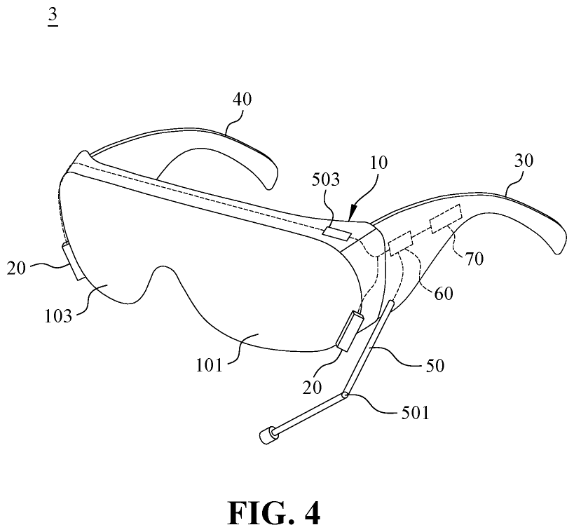

FIG. 4 is a schematic diagram illustrating a structure of a wearable device according to further embodiment of the present invention. Referring to FIG. 4, in this embodiment, the wearable apparatus 3 further includes a switch module 503, which is illustrated in FIG. 4 as being arranged on the frames 10, but it should be understood that the switch module 503 may be selectively arranged on the frames 10, the left temple 30 or the right temple 40, and is electrically connected to the power supply device 70 and the UV light source 20, such that the switch module 503 can control the detection device 50, the UV light source 20 on and off, or further, the switch module 90 can control whether the UV light source 20 should be irradiated together with the detection device 50.

For example, in other embodiments, the user 80 may turn on different irradiation modes of the UV light source 20 by pressing and holding the switch module 503, pressing switch modules 503 twice, or by other operation modes, for example, the UV light source 20 may be directly irradiated at a preset frequency, which may be any second in the range of 0.1 to 10 seconds, instead of irradiating with the detection device 50, so that the UV light source 20 forms a pulsed light source, thereby further achieving the purpose of low-power, power-saving, and efficient irradiation without losing the sterilizing effect.

It should be noted that, referring again to FIG. 1, in other embodiments of the present invention, the wearable device may function properly even if it does not include the detection device 50 and the control module 60, as described in the context of the priority of the present application, and reiterated herein. More in detail, in this embodiment, the UV light source 20 has been arranged at the lower left side of the left frame 101 and/or the lower right side of the right frame 103, and there is an included angle d1 between the UV light source 20 and the right side edge of the right frame 103, and there is an included angle d2 between the other UV light source 20 and the left side edge of the left frame 101, and the included angle d1 and the included angle d2 can be any angle in the range of 10-60 degree, so that the UV light source 20 irradiates the positions around, in front of, and above the mouth and the nose in the area A1 around the face of the user 80, so as to enhance the sterilization and disinfection effect against the positions around the mouth and nose of the user 80. Further, the UV light source 20 may be in a normally on state.

Further, if the control module 60 is added to the embodiment that does not include the detection means 50, the control module 60 is electrically connected to the power supply means 70 and the UV light source 20 as well, and the control module 50 controls the UV light source 20 to irradiate at a predetermined frequency, wherein the preset frequency is any second in the range of 1/15 second to 1/30 second.

In addition, if the switch module 503 is added to the embodiment that does not include the detection device 50, the switch module 503 is electrically connected to the power supply device 70 and the UV light source 20 as well, and the switch module 503 can control a switch of the UV light source 20. Further, the UV light source 20 may also be at least one deep UV light source.

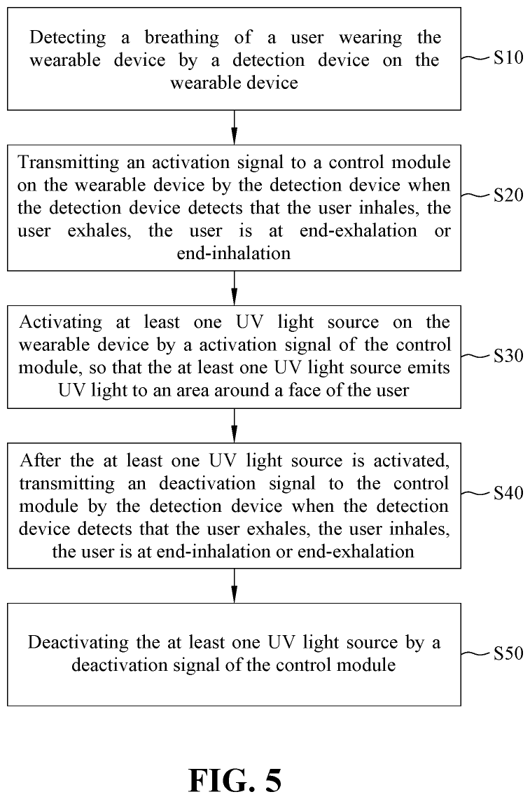

FIG. 5 is a flow chart illustrating an operation method of a wearable device in accordance with one embodiment of the present invention. Referring to FIGS. 1, 2a, 2b and 5, an operation method of the wearable device of the present invention comprises the steps of: step S10: detecting a breathing of a user 80 wearing the wearable device 1 by a detection device 50 on the wearable device 1; step S20: transmitting an activation signal to a control module 60 on the wearable device 1 by the detection device 50 when the detection device 50 detects that the user 80 inhales, the user 80 exhales, the user 80 is at end-exhalation or end-inhalation; step S30: activating at least one UV light source 20 on the wearable device 1 by a activation signal of the control module 60, so that the at least one UV light source 20 emits UV light to an area A1 (shown in FIG. 3b) around a face of the user 80; step S40: after the at least one UV light source is activated, transmitting an deactivation signal to the control module 60 by the detection device 50 when the detection device 50 detects that the user 80 exhales, the user 80 inhales, the user 80 is at end-inhalation or end-exhalation; and step S50: deactivating the at least one UV light source 20 by a deactivation signal of the control module 60, wherein the detection device 50 may be rotated by an adjustment mechanism 501, the at least one UV light source 20 may be at least one deep UV light source.

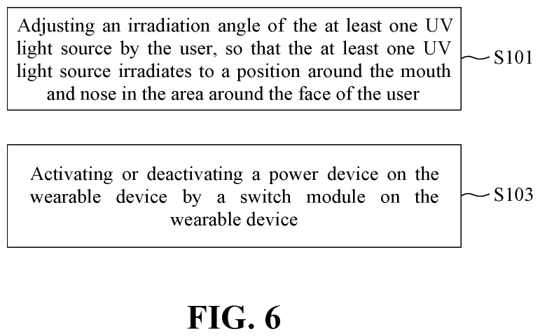

FIG. 6 is a schematic diagram illustrating an operation method of a wearable device according to another embodiment of the present invention. In another embodiment of the present invention, the operation method further comprises step S101 and step S103. With reference to FIGS. 2a, 2b, 3a, 3b, and 6, step S101 is: adjusting an irradiation angle of the at least one UV light source 20 by the user 80, so that the at least one UV light source 20 irradiates to a position around the mouth and nose in the area around the face of the user, to enhance the sterilization and disinfection effect against the positions around the mouth and the nose of the user 80 and eliminate bacteria or droplets remaining on the mask 90.

With reference again to FIGS. 2a, 2b, 4, and 6, step S103 is: activating or deactivating a power device on the wearable device 1 by a switch module 503 on the wearable device 1. Furthermore, the user 80 can turn on different irradiation modes of the UV light source 20 by pressing and holding the switch module 503, pressing switch modules 503 twice, or by other operation modes, for example, the UV light source 20 can be directly irradiated at a preset frequency, which may be any second in the range of 0.1 to 10 seconds, instead of irradiating with the detection device 50, so that the UV light source 20 forms a pulse light source, thereby further achieving the purpose of low-power, power-saving, and efficient irradiation without losing the sterilizing effect.

As can be seen the above, the present invention provides a wearable device and operation method thereof. In the wearable device of the present invention, the UV light source can efficiently sterilize and disinfect the surrounding area of the face in response to the user's breathing by the detecting device without keeping the UV light source in a normally on or normally off state, and the structure and operation method can also save power in the wearable device. Further, according to the present invention, the irradiation mode can be adjusted based on the user's health condition so that the UV light source can sterilize and disinfect the air inhaled or exhaled.

What is claimed is:

1. A wearable device comprising:
frames comprising a left frames and a right frame, the left frame being connected to the right frame by a bridge;
at least one ultraviolet (UV) light source arranged on the left frame and/or the right frame;
a left temple connected to the left frame;
a right temple connected to the right frame; and
a power supply device arranged in the frame, the left temple or the right temple, the power supply device being electrically connected to the at least one UV light source,
wherein the at least one UV light source emits UV light to an area around a face of a user,
wherein the at least one UV light source is arranged at a lower left side of the left frame and/or a lower right side of the right frame, and there is an included angle between the at least one UV light source and a left side edge of the left frame and/or a right side edge of the right frame, the included angle is any angle in the range of 10-60 degree, so that the UV light source irradiates to a position around a mouth and a nose in the area around the face of the user.

2. The wearable device according to claim 1, further comprising a control module, wherein the control module is arranged in the frames, the left temple or the right temple, the control module is electrically connected to the power supply device and the at least one UV light source, and the control module controls the at least one UV light source to irradiate at a preset frequency.

3. The wearable device according to claim 2, wherein the preset frequency is any second in a range of 1/15 second to 1/30 second.

4. The wearable device according to claim 1, further comprising a switch module, arranged on the frames, the left temple or the right temple, wherein the switch module is electrically connected to the power supply device and the at least one UV light source, and the switch module controls a switch of the at least one UV light source.

5. The wearable device according to claim 1, wherein the at least one UV light source is at least one deep UV light source.

6. A wearable device comprising:
frames comprising a left frame and a right frame, the left frame being connected to the right frame by a bridge;
at least one UV light source arranged at a lower left side of the left frame and/or a lower right side of the right frame;
a left temple connected to the left frame;
a right temple connected to the right frame;
a detection device arranged on the left frame, the right frame, the left temple or the right temple;
a control module arranged in the frames, the left temple or the right temple, and electrically connected to the detection device and the at least one UV light source, and
a power supply device arranged in the frames, the left temple or the right temple, and electrically connected to the at least one UV light source, the detection device and the control module,
wherein the detection device is used to detect breathing of a user of the wearable device, when the detection device detects that the user inhales, the user exhales or is at end-exhalation or end-inhalation, the detection device transmits an activation signal to the control module, the control module activates the at least one UV light source by the activation signal, so that the at least one UV light source emits UV light to an area around a face of the user,
wherein after the at least one UV light source is activated, when the detection device detects that the user exhales, inhales, the user is at the end-inhalation or end-exhalation, the detection device transmits a deactivation signal to the control module, and the control module deactivates the at least one ultraviolet light source by the deactivation signal.

7. The wearable device according to claim 6, wherein there is an included angle between the at least one UV light source and a left side edge of the left frame and/or a right side edge of the right frame, the included angle is any angle in the range of 10-60 degree, so that the UV light source irradiates to a position around a mouth and a nose in the area around the face of the user.

8. The wearable device according to claim 6, further comprising a switch module arranged on the frames, the left temple or the right temple, wherein the switch module is electrically connected to the power supply device, and the switch module controls a switch of the power supply device.

9. The wearable device according to claim 6, wherein the detecting device comprises an adjusting mechanism by which the detecting device rotates.

10. The wearable device according to claim 6, wherein the at least one UV light source is at least one deep UV light source.

11. An operation method of a wearable device comprising the steps of:
detecting a breathing of a user wearing the wearable device by a detection device on a wearable device;
transmitting an activation signal to a control module on the wearable device by the detection device when the detection device detects that the user inhales, the user exhales, or is at end-exhalation or end-inhalation;
activating at least one UV light source on the wearable device by a activation signal of the control module, so that the at least one UV light source emits UV light to an area around a face of the user;
after the at least one UV light source is activated, transmitting an deactivation signal to the control module by the detection device when the detection device detects that the user exhales, the user inhales, the user is at end-inhalation or the user is at end-exhalation; and
deactivating the at least one UV light source by a deactivation signal of the control module.

12. The operation method according to claim 11, further comprising a step of: adjusting an irradiation angle of the at least one UV light source by the user, so that the at least one UV light source irradiates to a position around a mouth and a nose in the area around the face of the user.

13. The operation method according to claim 11, further comprising a step of: activating or deactivating a power device on the wearable device by a switch module on the wearable device.

14. The operation method according to claim 11, wherein the detection device is rotated by an adjustment mechanism.

15. The operation method according to claim 11, wherein the at least one UV light source is at least one deep UV light source.
